(19) United States
(12) Patent Application Publication
(10) Pub. No.: US 2018/0185065 A1
(43) Pub. Date: Jul. 5, 2018

(54) IMPROVEMENTS RELATING TO BONE ANCHORS

(71) Applicants: NOTTINGHAM UNIVERSITY HOSPITALS NHS TRUST, Nottingham, Nottinghamshire (GB); THE UNIVERSITY OF NOTTINGHAM, Nottingham, Nottinghamshire (GB)

(72) Inventors: Donal Stewart MCNALLY, Nottingham, Nottinghamshire (GB); Bronek Maximilian BOSZCZYK, Nottingham, Nottinghamshire (GB)

(21) Appl. No.: 15/740,180

(22) PCT Filed: Jun. 30, 2016
(86) PCT No.: PCT/GB2016/051962

§ 371 Date: Dec. 27, 2017

(30) Foreign Application Priority Data
Jul. 2, 2015 (GB) 1511646.0

(51) Int. Cl. A61B 17/70; A61B 34/10; A61F 2/44

(52) U.S. Cl. CPC A61B 17/70; A61B 2017/00526; A61F 2/44; A61B 34/10

(57) ABSTRACT

The present invention describes a bone anchor having a bone abutment surface adapted for congruent attachment to a bone and methods for the production of said bone anchor. The manufacturing methods generally involve mapping of the form of the bone to which the bone anchor is to be applied, commonly carried out by means of an imaging technique. Manufacturing methods further include any suitable process used to make a three-dimensional object: such process generally include either additive or subtractive manufacturing methods. In particular, said bone anchors are useful for attachment to the spine. The present invention also provides a kit for use in spinal surgery, for correcting spinal deformities and fusing adjacent vertebrae in the spine, using the bone anchors described herein.

This invention relates to bone anchors, that is to say to anchorages for attachment to bones during corrective or other surgical procedures. The invention further relates to methods of producing such bone anchors, and to surgical methods involving their use.

The correction of spinal deformities such as congenital or degenerative scoliosis involves complicated surgical procedures in which rods are fixed to the spine. Early methods for correction of a curved spine involved the use of a straight rod attached to the spine on the convex side of the curve. Compression was used to correct the deformity in the coronal plane. Following that, the so-called “apical translation” technique was developed in which sublaminar (Luque) wires were used to attach the spine to straight rods. This technique allowed correction in the coronal and sagittal planes but still did not correct rotational deformity. Improved techniques were developed using curved rods which were fastened to the vertebrae at multiple points and then rotated to restore the desired sagittal profile.

Current techniques for the correction of spinal deformities involve the insertion of pedicle screws along adjacent vertebrae and the attachment of one or more rods. Correction is performed by segmental tightening of the screws at the apex of the curve, similar to tightening of sublaminar wires, and then direct vertebral derotation.

There has been considerable progress in the design of pedicle screws to facilitate the introduction and connection of the rod. For example, polyaxial screws may be used to facilitate the introduction of curved rods, contoured to the sagittal profile. Polyaxial screws with extended tabs have been developed to provide yet more freedom in bending the rod and placement of the rod in the unreduced screws: the extended tab is removed once the rod is secured. By tightening the screw set, the spine is pulled towards the rod segment-by-segment, resulting in a slow and gradual reduction of the deformity with load transfer through several screws. The polyaxial screws may be locked after introduction of the rod, to effectively turn them into monoaxial screws so that direct vertebral body rotation may be carried out, and then the assembly may be fine-tuned segment-by-segment using compression and/or distraction, if required.

Improvements in the locking design, the thread closure mechanism and the screw-rod interface are all current areas of development. Different size polyaxial and monoaxial screws are available, as well as low profile screws to treat small anatomies, cross-connectors to connect rods together, where two parallel rods are used, and rods made from different materials, with different lengths and different bending stiffnesses. Accessories such as awls, hooks, pedicle probes, ball tip feelers and bone taps are all available to assist in pedicle preparation. Different screwdrivers and wrenches to tighten the screws may be selected. These are all examples of the products that are being developed in this field to increase the breadth of options available to the surgeon for managing deformity cases.

Nevertheless, there are still considerable risks and disadvantages associated with the use of pedicle screws.

Poor screw placement is a major concern and can lead to pedicle fracture and/or nerve root impingement. It is estimated that 5-10% of all pedicle screws are misplaced, and that 0.2% of screw misplacements lead to significant neurological deficit. An alternative to pedicle screw fixation or one that reduces the risk of screw misplacement is therefore very attractive.

Unfortunately, the problem is not solved simply by using smaller screws. A reduction in screw size reduces the strength of attachment of the screw in the pedicle and as a result the screw is not always effective. This has been seen in the correction of idiopathic scoliosis in adolescent girls, as in many cases they have small pedicles and therefore smaller screws must be used to avoid hitting the spinal cord.

There is also a particular need for an alternative fixation system for the osteoporotic spine, because fixation of the screw is more likely to fail if the quality of the bone is poor.

Other technologies that have been developed to solve the problem of screw misplacement include complex video-guided navigation systems, which are expensive and require significant operating theatre time to register the patient position to the computer model. X-ray guided screw placement is the current standard of care, but its main disadvantages are that it involves significant radiation doses to the patient and increases the surgeon's radiation exposure. More importantly, it does not eliminate screw misplacement. Detection systems to identify breached pedicles help to minimise neurological complications but do not prevent damage and therefore weakening of the pedicle wall. Custom-manufactured drill guides are available, but are not used widely at present, mainly because of the level of planning required prior to surgery.

Hooks provide one alternative to pedicle screws, and may be used in addition to the screws. However, there is evidence that lumbar pedicle screws offer greater curve correction, better maintenance of correction and improved pulmonary function compared with hook instrumentation in the treatment of adolescent idiopathic scoliosis.

All known systems such as those described above involve technically demanding procedures that present a risk of serious injury to the patient. Those procedures require the surgeon to be thoroughly knowledgeable not only in the medical and surgical aspects of the implant, but also the mechanical and metallurgical limitations of metallic surgical implants. Postoperatively, the patient must take extreme care in terms of weightbearing and stresses applied to the implanted devices until healing is complete. Non-compliance by the patient with postoperative instructions may lead to failure of the implant and the possible need for additional surgery to remove the device.

There is now provided an improved device for attachment to bone which overcomes and/or substantially mitigates problems associated with the prior art.

According to the invention there is provided a bone anchor having a bone abutment surface adapted for congruent attachment to a bone.

By “congruent attachment” in the context of the present invention is meant that the abutment surface of the bone anchor has a form that matches substantially exactly the form of the bone surface to which the bone anchor is to be attached. In other words, the surface of the bone and the bone abutment surface of the bone anchor coincide substantially exactly when superimposed, the relief profile of the bone abutment surface being in effect a negative reproduction of the bone surface (ie having rececces and protrusions that match protrusions and recesses respectively on the bone surface). Thus, “matches substantially exactly” means that, at least to the extent permitted by the method by which the bone anchor is manufactured, the form of the bone abutment surface mirrors the form of the bone to which the bone anchor is to be applied.

The bone abutment surface is therefore normally non-planar, and generally will not have a regular geometric form, instead having a complex form adapted to provide a close fit between the bone anchor and the bone to which it is, in use, applied.

It will be appreciated that, whilst in many cases, the close fit may be between substantially the whole undersurface of the bone anchor and the bone, in other cases the congruent fit may be between only part of the undersurface of the bone anchor and the bone.

A consequence of the congruent nature of the bone abutment surface and the surface of the bone to which the bone anchor is applied is that, when adhesive is interposed between those surfaces, the spacing between them (ie the thickness of the adhesive) is substantially uniform.

In order to further ensure that the layer of adhesive is of uniform and appropriate thickness, the bone abutment surface may be provided with spacer formations that directly contact the bone to which the bone anchor is applied, and which position the bone abutment surface at precisely the correct separation from the bone surface.

Such spacer formations may, for instance, take the form of one or more downwardly depending projections from the bone abutment surface. Such a projection may be a downwardly depending rim formed at or near the periphery of the bone abutment surface. Alternatively, or in addition, such a projection may be a pin or pillar formed on the bone abutment surface.

In general, the thickness of the adhesive layer (and hence the depth of any spacer formations) will be rather small, generally less than 1 mm and more commonly less than 200 μm. Typically, the desired thickness of the adhesive layer will be in the range 50-125 μm, and commonly 80-100 μm. The optimum thickness of the adhesive layer will depend on a number of factors, including the particular bone to which the bone anchor is to be applied, the quality of that bone, the size of the bone anchor, and the properties of the particular adhesive used.

It has been found that a bone anchor having a bone abutment surface adapted for congruent attachment can be affixed more securely and/or more readily and/or more safely to a bone than is the case for conventional bone anchors. In particular, the bone anchor may be attached to the bone by means of adhesive, rather than by means of mechanical fasteners such as screws. Because the bone abutment surface matches the contours of the bone to which it is applied, adhesive interposed between the two has a substantially uniform thickness, leading to a strong bond. In some circumstances, it may be necessary or desirable to employ mechanical fasteners, either instead of or in addition to adhesive, but in such cases the congruent fit of the bone anchor with the bone enables specific screw trajectories to be predetermined, which direct the screw through the pedicle with minimal risk of malplacement. Moreover, the close abutment of the bone anchor with the underlying bone may enable the use of smaller fasteners, ie most typically smaller screws. This in turn leads to greater ease of fixation and/or a reduction in the risk of pedicle fracture or neurological damage to the patient.

In order for the bone abutment surface of the bone anchor to have a congruent fit with the underlying bone, it will generally be necessary for the form of the underlying bone to have been determined and for the bone anchor to have been manufactured with a corresponding bone abutment surface. Thus, in general the bone anchors of the invention will be custom-made for individual patients.

The process of manufacture of the bone anchor will therefore generally involve mapping of the form of the bone to which the bone anchor is to be applied, and this will most commonly be carried out by means of an imaging technique. Any suitable imaging technique may be used, examples being computer tomography (CT), magnetic resonance imaging (MRI) and ultrasound imaging. In some instances, a combination of such imaging techniques may be used. Normally CT, or MRI with CT provide the clearest resolution of bone to tissue.

In computer-tomography, computer processing is used to produce tomographic (cross-sectional) images of a scanned object, and thus a three-dimensional image of the inside of a scanned object, from a large series of two-dimensional radiographic images taken from different angles. X-ray CT is currently the most common form of CT in medicine, although other types exist, such as positron emission tomography and single-photon emission tomography.

Magnetic resonance imaging is a medical imaging technique that uses magnetic fields and radio waves to form images of the body. In most medical applications, protons in tissues containing water molecules are used to create a signal that is processed to form an image of the body. To produce an image, the patient is positioned in the MRI scanner which forms a strong magnetic field around the area to be imaged. First, energy from an oscillating magnetic field is temporarily applied to the patient at the appropriate resonance frequency, and the excited hydrogen atoms emit a radio frequency signal which is measured by a receiving coil. The radio signal can be made to encode position information by varying the main magnetic field using gradient coils. The contrast between different tissues is determined by the rate at which excited atoms return to the equilibrium state as the gradient coils are rapidly switched on and off.

Ultrasound imaging (also referred to as sonography or ultrasonography) is an imaging technique that usually involves the transmission of a pulse of ultrasound into the body using an ultrasound transducer. The sound reflects from structures within the body, and these reflections are recorded and used to construct an image.

Whichever method of imaging is used, the output will generally be a data file that defines the form of the bone surface to which the bone anchor is to be applied. That data file may then be utilised in the process of manufacture of the bone anchor.

The bone anchor of the invention may be manufactured by any suitable process. In general, suitable processes are categorised as either additive or subtractive manufacturing methods.

Additive manufacturing is any of various additive processes used to make a three-dimensional object, including 3D printing, extrusion and sintering-based processes. In general, such processes involve laying down successive layers of material under computer control to produce a three-dimensional object. Numerous additive manufacturing processes are now available. These processes differ from each other in the way that layers are deposited and in the materials that are used. In some methods, eg selective laser melting (SLM) or direct metal laser sintering (DMLS), selective laser sintering (SLS), fused deposition modeling (FDM), and fused filament fabrication (FFF), material is melted or softened to produce the layers, while in others, eg stereolithography (SLA), liquid materials are cured to form a solid.

One particular additive manufacturing method that may be used in the manufacture of a bone anchor of the invention is fused deposition modeling (FDM), in which the bone anchor is produced by extruding small beads of material which harden immediately to form layers. Material in the form of a thermoplastic filament or metal wire is supplied to an extrusion nozzle. The material is heated by the nozzle and the flow of material is turned on and off under the control of a computer or microprocessor, which also controls the movement of the nozzle relative to the object being produced.

In FDM, various polymers may be used, including acrylonitrile butadiene styrene (ABS), polycarbonate (PC), polylactic acid (PLA), high density polyethylene (HDPE), PC/ABS, polyphenylsulfone (PPSU) and high impact polystyrene (HIPS).

Another particular additive manufacturing method that may be used in the invention is the selective fusing of materials in a granular bed. Such techniques involve fusing of parts of the layer and then downward movement in the working area, adding another layer of granules and repeating the process until the piece has built up. In such a process, unfused media can be used to support overhangs and thin walls in the component being produced, reducing the need for temporary auxiliary supports, as may be required in FDM. The granular material is typically sintered into a solid using a laser. Examples of this kind of process include selective laser sintering (SLS), which may use metals or polymers (eg polyetheretherketone (PEEK), polyamide (PA), polystyrene (PS) and others), and direct metal laser sintering (DMLS).

In another process, selective laser melting (SLM), the powder granules are not sintered, but are completely melted using a high-energy laser. Components produced in this way have greater density than sintered materials, and have mechanical properties similar to those of conventional manufactured metals.

Another additive manufacturing method that may be particularly useful in the invention is electron beam melting (EBM), in which metal is melted layer by layer using an electron beam in a high vacuum. EBM components are free of voids, dense and of high strength. Metals that can be used include titanium alloys.

Subtractive manufacturing is any of various processes in which a piece of raw material is cut into a desired shape and size by controlled material removal. Such processes are generally referred to as machining, and are most commonly used for the shaping of components in metal. For example, multi-axial milling is a process in which a three-dimensional shape is milled out of a solid block of material. Bone anchors of the invention may be prepared by such methods, eg in medical grade metals such as titanium or titanium alloys, or in polymeric materials.

The manufacturing methods described above may be used to produce the bone anchor itself directly. Alternatively, such a method may be used to produce a mould that may then be used to produce a bone anchor by another conventional process, eg casting in metal, or injection moulding.

Thus, according to a second aspect of the invention there is provided a method for the production of a bone anchor, which method comprises:
a) generating a data file embodying the form of the bone to which the bone anchor is to be attached; and
b) manufacturing the bone anchor with a bone abutment surface formed in accordance with the data file, such that the abutment surface is adapted for congruent attachment to the bone.

As noted above, the date file will most commonly be the output of an imaging procedure.

The two steps in the production of the bone anchor, ie generation of the data file and subsequent manufacture of the bone anchor utilising that data file, may, and in most cases will be, carried out by different parties. In order for the data file to be generated, the patient will normally attend an imaging clinic where they will be scanned in accordance with instructions from the patient's clinician, in particular in relation to the areas of bone, eg the particular vertebrae, that are to be scanned. Once the data file has been generated, it will be forwarded to a manufacturing facility, which may be associated with the imaging clinic but more commonly would be an independent manufacturing organisation. The bone anchor is then produced by the manufacturer and supplied to the patient's clinician or healthcare provider for use in surgery. The hospital at which the bone anchor is used in surgery may be, but is not necessarily, the same as or associated with the clinic at which the patient was scanned in order to generate the data file.

Thus, the invention also provides a method for the production of a bone anchor, which method comprises
manufacturing the bone anchor with a bone abutment surface formed in accordance with a data file embodying the form of the bone to which the bone anchor is to be attached, such that the abutment surface is adapted for congruent attachment to the bone.

The bone anchors of the invention will generally be formed with attachment parts by which they are coupled to other components. Those attachment parts may have various structures, as described in more detail below. The choice of the most appropriate attachment part may be made by the surgeon shortly before, or during, the surgical procedure. It may therefore be beneficial for the surgeon to have at his disposal a range of bone anchors with identical bone abutment surfaces but alternative attachment parts. Therefore, once the data file has been generated, the manufacturer may produce and supply sets of alternative bone anchors for each area of bone to which an anchor is to be fitted, the choice of which member of that set is used being made subsequently by the surgeon.

Thus, according to another aspect of the invention, there is provided a plurality of bone anchors having bone abutment surfaces adapted for congruent attachment to the same area of a patient's bone, the bone anchors being provided with differing attachment parts for coupling of the bone anchor to other components.

As noted above, the bone anchor may be fixed to the bone using a suitable adhesive. The congruent fit of the bone anchor against the bone means that adherence is strong.

The adhesive may be any biocompatible adhesive which is also compatible with the material that the anchor is manufactured from. Examples include acrylate and methacrylate adhesives such as cyanoacrylate. Suitable adhesives may include adhesives used for dental purposes such as the fixing of dental crowns, including those referred to as dentine (or dentin) bonding agents. These include, but are not limited to, glass-ionomer composites. Other adhesives that may be suitable include bone cements, such as that known as Kryptonite™ bone cement.

Thus, according to another aspect of the invention, there is provided a plurality of bone anchors having bone abutment surfaces adapted for congruent attachment to the same area of a patient's bone, the bone anchors being provided with differing attachment parts for coupling of the bone anchor to other components.

As discussed above, such a method may, and generally will, involve the preliminary steps of using an imaging technique to generate a data file embodying the form of the bone to which the bone anchor is to be attached, and subsequently manufacturing the bone anchor with a bone abutment surface formed in accordance with the data file.

Thus, in certain embodiments, the invention provides a surgical method, which method includes the step of affixing to a bone a bone anchor having a bone abutment surface adapted for congruent attachment to the bone,
wherein the bone anchor has been manufactured by a process including the steps of
   a) using an imaging technique to generate a data file embodying the form of the bone to which the bone anchor is to be attached; and
   b) manufacturing the bone anchor with a bone abutment surface formed in accordance with the data file, such that the abutment surface is adapted for congruent attachment to the bone.

Although the bone anchor of the invention may be used in a wide variety of applications, the areas in which it is currently envisaged that the bone anchor will be most valuable are spinal fusion and the correction of spinal deformities. The anchors provide an alternative to the current fixation system of pedicle screws and afford a number of significant advantages.

Thus, according to a further aspect of the invention there is provided a method for correcting spinal deformities, which method includes the steps of affixing bone anchors to at least two vertebrae and then connecting the bone anchors to support the spine,
wherein the bone anchors have bone abutment surfaces adapted for congruent attachment to the at least two vertebrae, and
wherein the bone anchors have been manufactured by a process including the steps of
   a) using an imaging technique to generate one or more data files embodying the form of the at least two vertebrae to which the bone anchors are to be attached; and
   b) manufacturing the bone anchors with bone abutment surfaces formed in accordance with the one or more data files, such that the abutment surfaces are adapted for congruent attachment to the surface of the at least two vertebrae.

According to another aspect of the invention there is provided a method for fusing adjacent vertebrae in the spine, which method includes the steps of affixing bone anchors to at least two adjacent vertebrae and then connecting the bone anchors to fuse the vertebrae,
wherein the bone anchors have bone abutment surfaces adapted for congruent attachment to the at least two vertebrae, and
wherein the bone anchors have been manufactured by a process including the steps of
   a) using an imaging technique to generate one or more data files embodying the form of the at least two vertebrae to which the bone anchors are to be attached; and
   b) manufacturing the bone anchors with bone abutment surfaces formed in accordance with the one or more data files, such that the abutment surfaces are adapted for congruent attachment to the surface of the at least two vertebrae.

The fact that the bone anchors and methods of the present invention do not necessarily require the use of screws for secure attachment of the bone anchors to the bone represents a major breakthrough in this field, with numerous clear advantages. The invention may remove the need to drill into the bone, and hence may reduce or eliminate the risk of neurological damage due to screw misplacement and the risk of pedicle fracture to anterior vascular systems. As noted above, even where screws are used, those screws may be smaller than would otherwise be required, again leading to a reduction in risk to the patient.

When additional pedicle screws are required, the bone anchors of the present invention also enable intraoperative robotics to be used, which have the potential to improve the accuracy and safety of pedicle screw placement even further. The use of robotic devices is of great interest in this area, and several robotic applications exist for navigating either drill guides or drills for the insertion of pedicle screws into the spine. However, there are serious issues with the current technology because, until now, the robot could not determine and adjust its position relative to the vertebra without the risk of error. In particular, current means of attaching reference points to the spine for active or passive navigation are not satisfactory, so the reference points are reasonably easy to manipulate or move, misleading the robotics' true trajectories. In addition, movements of the body cause a risk of pedicle screw misplacement due to the possibility of the spine moving away from the tool guided by the robot. There is an inherent phase lag between any control mechanism that monitors the position of the individual vertebra and the advancing tool.

The bone anchors of the present invention attach firmly to the spine, thus allowing for the manufacture of drill guides and clearly defined trajectories for pedicle screw insertion which do not move on the vertebra. Furthermore, they provide a firm anchor point which may be manipulated by a robot, thus allowing robots to be used to manipulate the vertebra into the desired position for deformity correction. Robots may be used to manipulate the vertebra during spinal surgery, or to attach connectors or rods or the like, whether additional pedicle screws are required or not.

By having such a firm attachment point to which a robotic arm can attach to each vertebra it is now possible to not only manipulate the vertebra but to constantly assess the attachment of the bone anchor to the vertebra through the resistance provided by the adhesive. Furthermore, surface recognition of the individual bone anchor may be possible through etching or similar visual marking on the surface enabling the robot to determine the position of the specific bone anchor and therefore the attached vertebra and space. The robot may subsequently utilise the predetermined screw trajectory guide for drilling into the pedicle and inserting the preoperatively determined screw size. Once this has been achieved the robot may be able to manipulate individual vertebra via the bone anchors into the desired position. The robot “knows” the location of each vertebra relative to the others, along with the spatial orientation of the bony details of each vertebra. The means that it knows the shape of the individual vertebra for insertion of screws etc accurately, and also knows the position of the vertebrae relative to each other for the purpose of manipulation and the changing the shape of the spine through movement of the vertebrae relative to each other. As the vertebra may be manipulated individually, an algorithm may be developed that would prevent excessive translation between the vertebrae to take place that could injure the spinal cord through shear or distraction. This may provide a degree of safety that cannot be achieved manually. Once deformity correction has been achieved, the fastening devices such as rods may be inserted.

The bone anchors of the present invention are designed for each patient based on imaging data. The design process can be optimised for manufacture, in particular additive manufacture. Attachment of the bone anchors may be via direct gluing of congruent surfaces, and so compared to pedicle screw systems, for example, surgery may be less time-consuming and simpler to perform, and the patient's recovery time may consequently be reduced.

As noted above, because the bone abutment surface of the bone anchor has a congruent fit to the bone to which the bone anchor is applied, the strength of adhesion is good, even where adhesion is achieved solely through the use of adhesive. A bone anchor according to the present invention with a surface area of approximately 2 cm² is expected to have a bond strength of about 800-1200N on all bone types. This meets the demands required for its application in the correction of spinal deformities and outperforms pedicle screws in osteoporotic bone (for which the pull out strength may be only 200-500N).

Because the bone anchors of the present invention are custom-made for individual patients, on demand and as required, the clinic does not need to maintain a stock of large numbers of bone anchors of various sizes and forms. In contrast, pedicle screws and other related equipment need to be stored, and multiple options (for example different sizes, monoaxial and polyaxial types, different angles etc) must be available to the surgeon during surgery.

As described above, once the data file has been generated, the manufacturer may produce and supply sets of alternative bone anchors for each area of bone to which an anchor is to be fitted, the choice of which member of that set is used being made subsequently by the surgeon. The most significant production costs are generated during the initial design of the bone abutment surface and (though probably to a lesser extent) during manufacture of the first unit, because of the tooling and/or programming required to set up manufacture. However, once the first unit has been made, the cost of producing additional units is relatively small (due to the small amount of material required), and as a consequence, it is practicable to produce several anchors of varying design for attachment to the same area of bone, so that the surgeon can select the most appropriate at the time of surgery. This eliminates the need for the surgeon to be directly involved in the production of the devices (other than to specify an appropriate data set, for example, and to specify the areas to be treated).

Once the bone abutment surface of the bone anchor has been designed to fit the corresponding surface of the bone, the rest of the anchor may have any desired form (subject only to limitations imposed by the need for the bone anchor to be implantable in the patient).

In particular, the bone anchors may include any of a variety of different attachment parts. To facilitate the design process, the attachment parts may be chosen from a standard library of such parts. Different attachment parts may include fixation points for spinal instrumentation including posterior rods (for deformity correction and fusion), for example ball or eyelet connections, and attachments for facet joint replacement or augmentation, or dynamic stabilisation.

Other suitable attachment parts include bushings to guide drilling, tapping and/or insertion of a pedicle screw, and posts that serve as additional pre-screw drill guides. The bone anchor may include locking features to provide a mechanical interlock between the anchor and the screw, permitting sharing of loading.

A practical approach envisaged by the inventors is to manufacture four different variants of each bone anchor for selection by the surgeon prior to or during surgery. The variants of most interest are currently the following:
an anchor with an eyelet for connection to posterior rods using a cord and universal clamp;
an anchor with ball connection onto which a standard polyaxial tulip clamp can be fitted by the surgeon—this type of tulip clamp fitting is common in standard pedicle screw fixation systems;
an anchor with a bush to guide pedicle screw placement and locking features to form a combined glue-screw fixation—there are a number of different locking mechanisms currently used to lock bone screws to fracture fixation plates that may be employed;
a bone anchor with a removable drilling and/or tapping guide for conventional pedicle screw fixation—disposable drill guides are currently available but not commonly used, due to the level of planning and the requirement for intraoperative radiographs before surgery.

The bone anchors may also be formed with porous areas, for example on the dorsal or medial surface. Such porous regions may allow for ingress of adhesive into the bone anchor, thereby improving adhesion, or may allow for ingress of bone or other tissue. Hydroxyapatite (or other osteoconductive) coatings may also be used, or cytokines, such as bone morphogenetic protein (BMP), to facilitate bony secondary fixation.

To aid the surgeon and to reduce the risk of errors during surgery, the bone anchors may be manufactured with surface features that constitute labelling, for example indicating the number of a vertebra to which the bone anchor fits and the side (left or right), or the name or other identifier for the patient.

In many cases, where they are to be used in spinal surgery, the bone anchors of the invention will be produced in pairs, the members of the pair being for attachment to the right and left pedicles of a vertebra. However, the bone anchor may alternatively be a single component that extends across both pedicles of the vertebra. Bilateral bone anchors may be advantageous where additional strength is required, for example to support osteoporotic bone. Such a bilateral bone anchor may be provided with a pair of attachment parts, so that the anchor is coupled to other components in a manner similar to the way in which those components would be coupled to a pair of bone anchors affixed to the right and left pedicles. Alternatively, the bilateral bone anchor may be provided with a single attachment part, most commonly centrally positioned, for example to allow for the attachment of a single, central rod, rather than two rods on either side of the spinal cord. Such an arrangement is believed to be novel, and the invention thus further provides a kit for use in spinal surgery, the kit comprising at least two bone anchors, each having a bone abutment surface adapted to have a congruent fit with a corresponding vertebra of a patient's spine, the bone anchors extending when applied to those vertebrae across both pedicles of the vertebrae, and the bone anchors being provided with attachment parts disposed centrally and adapted for coupling to a rod disposed, in use, substantially centrally of the patient's spine.

The invention will now be described in greater detail, by way of illustration only, with reference to the accompanying drawings, in which:

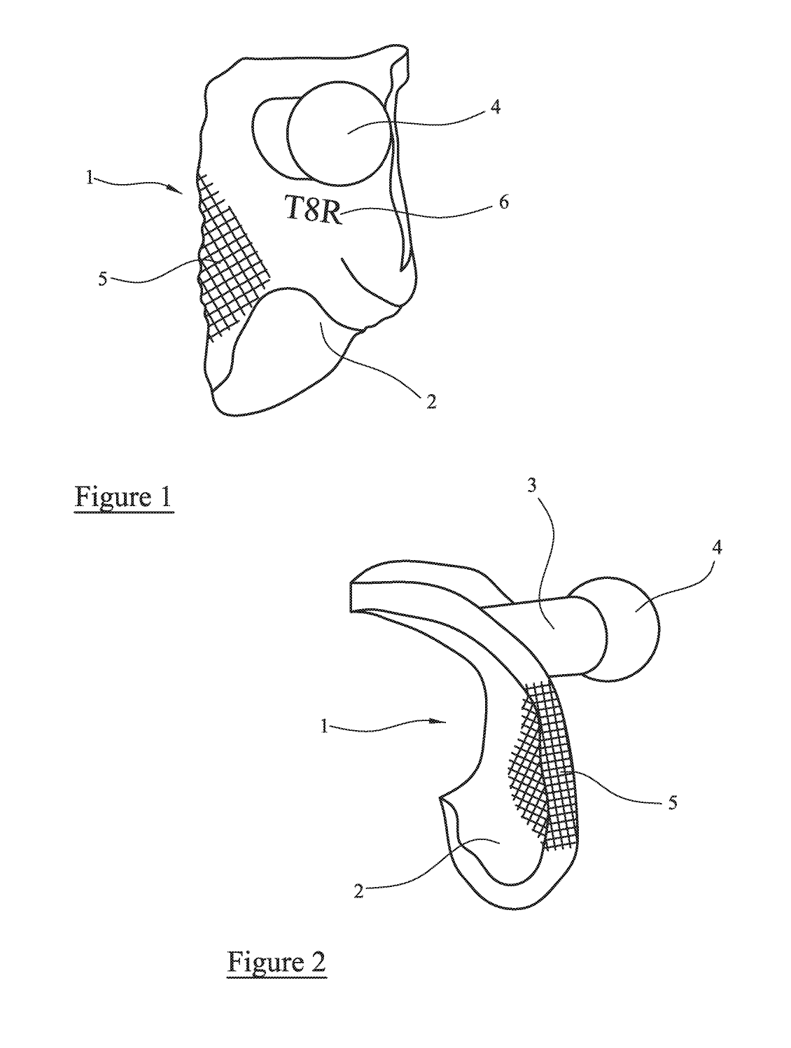

FIG. 1 shows a first embodiment of a bone anchor according to the invention, with a ball connection;

FIG. 2 is a side view of the bone anchor of FIG. 1;

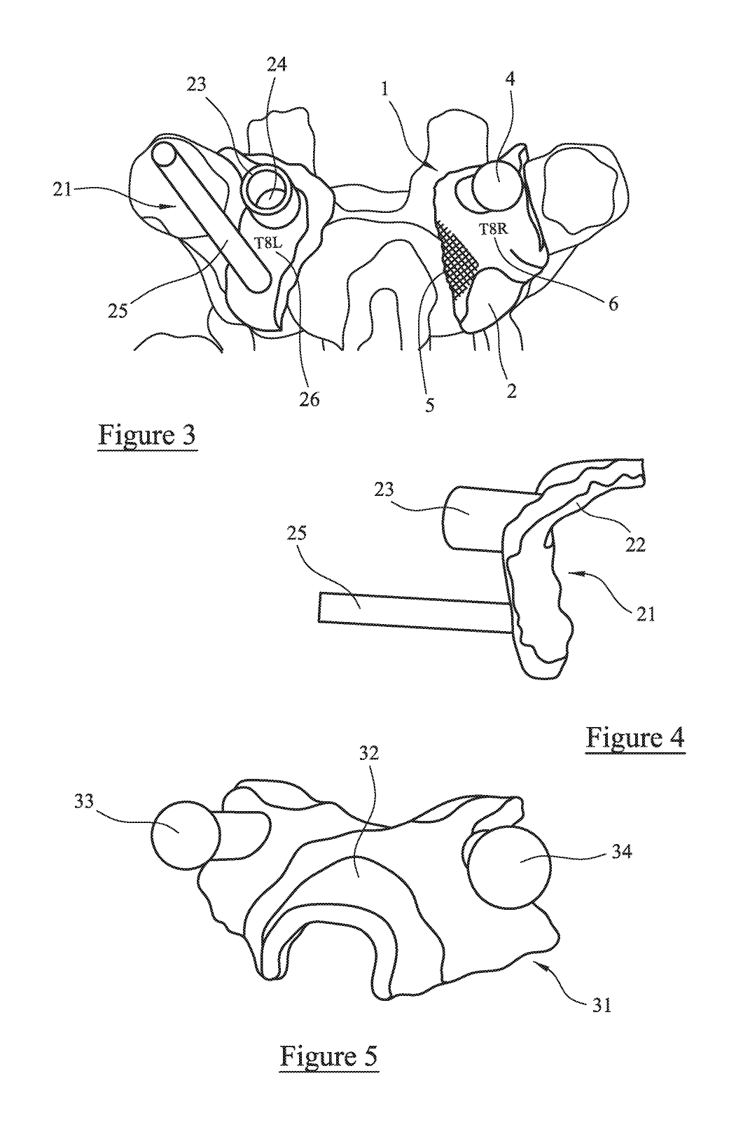

FIG. 3 shows the bone anchor of FIG. 1 applied to the right pedicle of a vertebra, and a second embodiment of a bone anchor according to the invention, with a drill guide and bushing for insertion of a pedicle screw, applied to the left pedicle;

FIG. 4 is a side view of the second embodiment of the bone anchor;

FIG. 5 shows a third embodiment of a bone anchor according to the invention, being a bilateral anchor applied to both pedicles of a vertebra;

FIG. 6 is a side view of the bilateral bone anchor of FIG. 5;

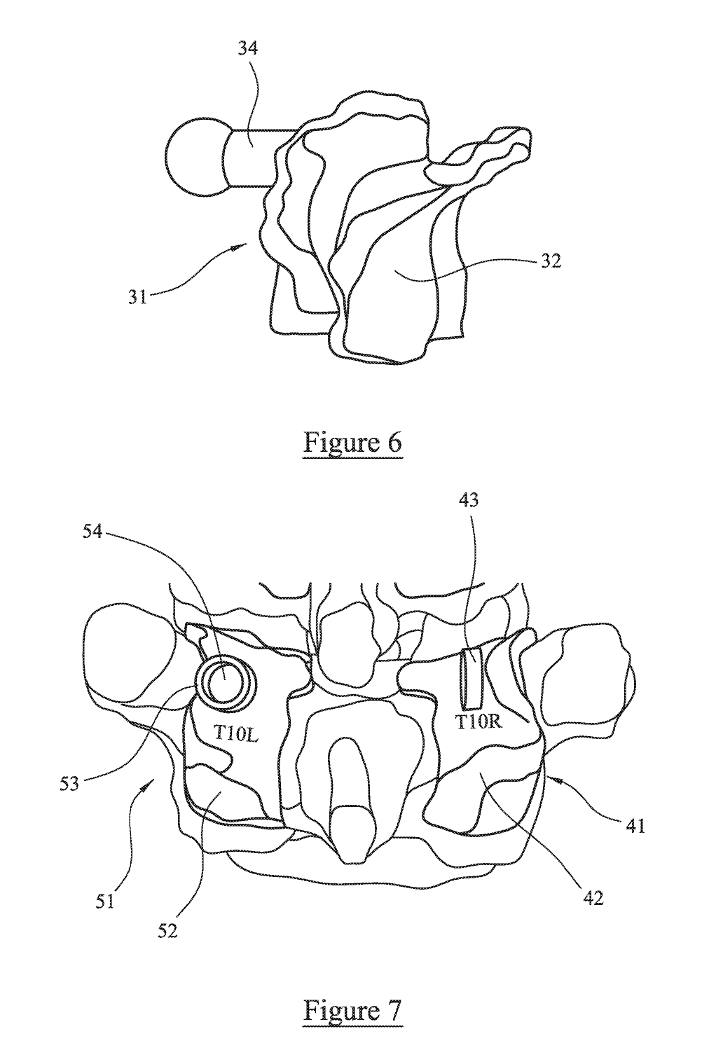

FIG. 7 is a view similar to FIG. 3, but showing a fourth embodiment of a bone anchor, with an eyelet connection, applied to the right pedicle, and a fifth embodiment of a bone anchor, with a bushing for a pedicle screw, on the left pedicle;

FIG. 8 is a side view of the fourth embodiment of the bone anchor, with an eyelet connection;

FIG. 9 is a side view of the fifth embodiment of the bone anchor, with a bushing for a pedicle screw;

FIG. 10 is a schematic illustration of the manner in which the bone anchor of FIG. 5 may be produced by an additive manufacturing process;

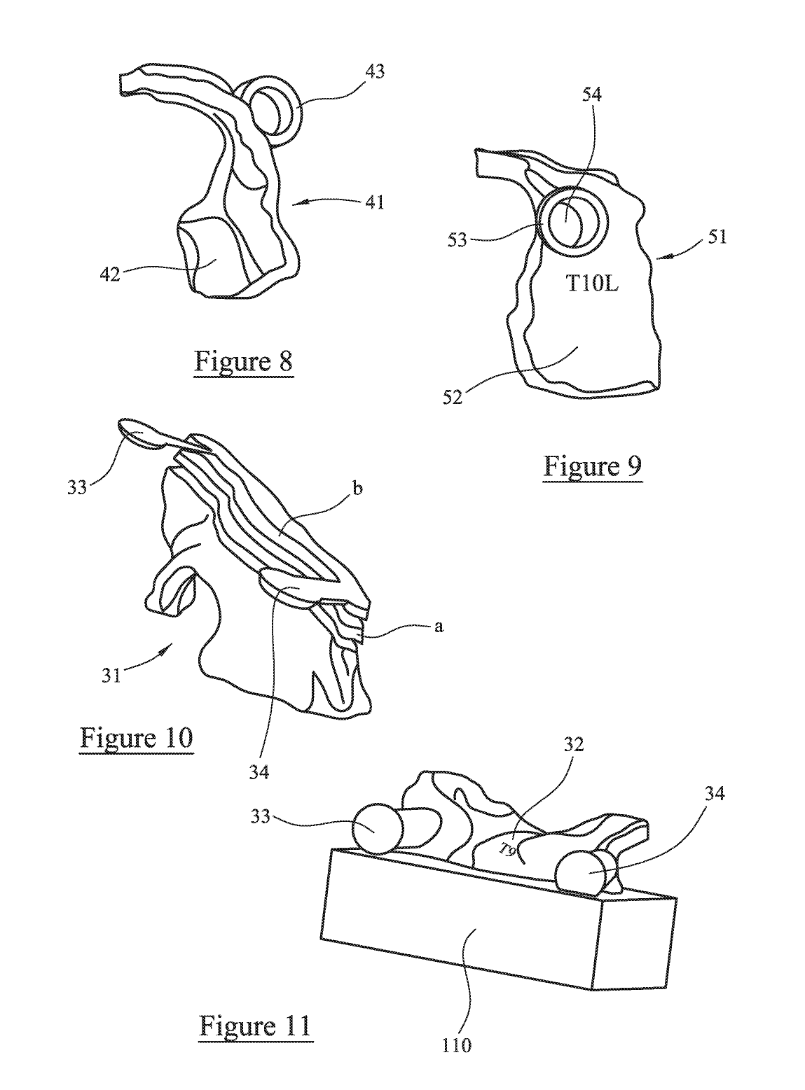

FIG. 11 is a schematic illustration of the manner in which the bone anchor of FIG. 5 may be produced by a subtractive manufacturing process; and

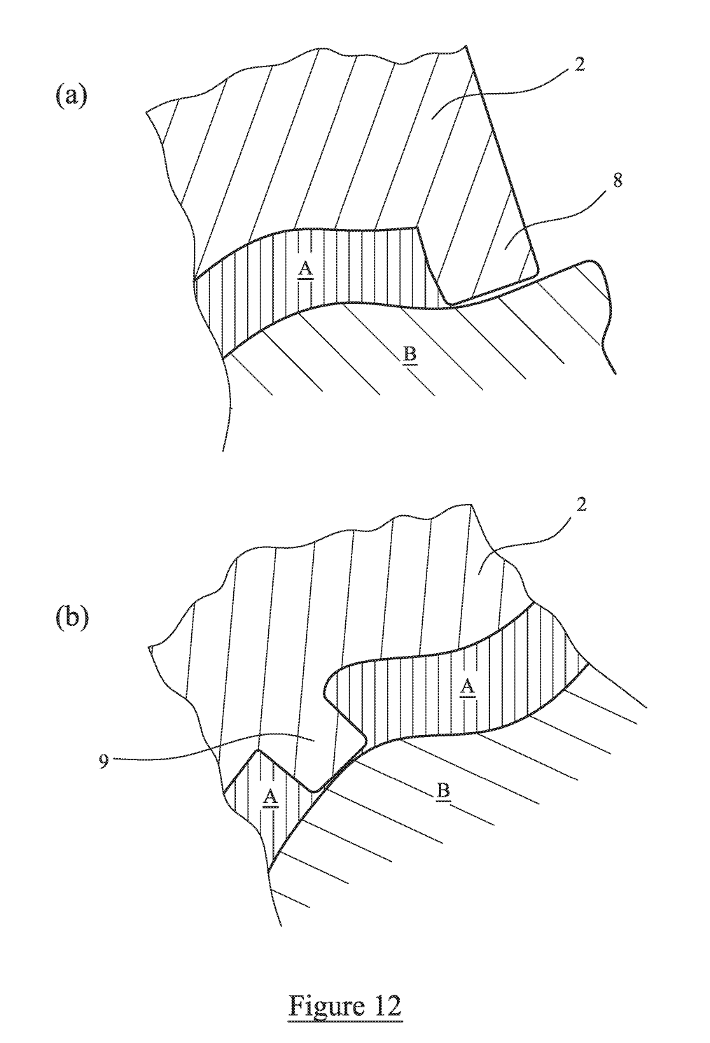

FIGS. 12(a) and 12(b) illustrate schematically two different types of spacer formation that may be incorporated into a bone anchor of the invention.

FIGS. 1 and 2 show a first embodiment of a bone anchor according to the invention, generally designated 1. The bone anchor 1 is intended for application to a patient's spine for use in the correction of a spinal deformity. The same is true of the other embodiments described below.

The bone anchor 1 comprises a baseplate 2 which is custom-manufactured for congruent attachment to a patient's vertebra. The baseplate 2 has a generally concave undersurface that is shaped to fit against the pedicle of the vertebra with a congruent fit. The baseplate 2 has an approximately uniform thickness.

An upstand 3 with a ball-shaped end 4 projects from the upper surface of the baseplate 2, and serves for attachment of the bone anchor 1 to other components used in corrective spinal surgery, eg rigid rods or wires or the like. The baseplate 2 is also formed with a porous region 5 of open structure. This allows for ingress of tissue into the bone anchor 1, thereby leading to enhanced fixation of the bone anchor 1. The baseplate 2 also carries an integrally formed label 6, being the code “T8R”, which indicates that the bone anchor 1 is designed to fit vertebra T8 on the right pedicle, as shown in FIG. 3.

The bone anchor 1 is made from titanium alloy (Tialloy), typically by an additive manufacturing process.

To adhere the bone anchor 1 to the vertebra, a suitable medical grade adhesive is applied to the undersurface of the baseplate 2 and the bone anchor 1 is pressed into place. As can be seen in FIG. 3, when the bone anchor 1 is positioned on the pedicle of the vertebra, the upstand 3 extends substantially perpendicular to the patients's spine. At a suitable point during surgery, a rod or the like (not shown) may be attached by the surgeon to the ball-shaped end 4 of the upstand 3 by means of a suitable coupling component, such as a tulip clamp.

FIG. 3 shows a second embodiment of a bone anchor according to the invention, generally designated 21, attached to the left pedicle of the vertebra that also carries the first embodiment 1 on the right pedicle. This embodiment 21 is also shown in FIG. 4. It should be understood that this arrangement is for illustration only; in most instances, where a bone anchor such as the first embodiment 1 is affixed to one pedicle, a second bone anchor of similar form will be attached to the other pedicle.

The bone anchor 21 has a baseplate 22 of generally similar form to that of the first embodiment 1, save that in this case there is no porous region. The upper surface of the baseplate 22 is formed with two upstanding formations: a bushing 23 around a circular opening 24 in the baseplate 22, and a post 25. As for the first embodiment 1, the baseplate is formed with a label 26, “T8L”, which indicates that it is made to fit vertebra T8, on the left pedicle.

In this second embodiment, the bone anchor 21 is attached to the bone using a pedicle screw, in addition to adhesive. The post 25 is sized and angled to guide a drill, with the drill bit moving through the bushing 23 and opening 24 to form a hole in the pedicle, the orientations of the post 25 and bushing 23 being such that the hole is formed at the optimal position and orientation. The bushing 23 also serves to guide the drill, and potentially the depth of the hole, and then to act as a guide for insertion of the pedicle screw.

The post 25 is designed to be removed from the baseplate 22 once it has been used to guide the drill and/or insertion of the pedicle screw, and the bushing 23 may also be designed to be removed after use, for example by unscrewing.

Thus, the bone anchor 21 is attached to the left pedicle of vertebra T8 using adhesive, a hole is drilled, guided by the post 25 and bushing 23, and a pedicle screw (not shown) is then inserted. The post 25 and bushing 23 are then removed.

FIGS. 5 and 6 show a third embodiment of a bone anchor according to the invention, generally designated 31. This embodiment is termed “bilateral”, by which is meant that it has a single baseplate 32 that is applied across both the right and left pedicles of a vertebra. The bone anchor 31 has two ball-headed upstands 33,34 that extend from the baseplate 32 in a similar fashion to the upstand 3 of the first embodiment 1.

Thus, when the bone anchor 31 is applied to a vertebra, the upstands 33,34 extend generally perpendicularly from opposite sides of the patient's spine. Two or more such bone anchors 31 may then be connected by rods coupled to the ball-headed upstands 33,34 by any suitable means, eg tulip clamps.

Finally, FIG. 7 shows fourth and fifth embodiments of a bone anchor according to the invention, generally designated 41 and 51 respectively. These embodiments are shown separately in FIGS. 8 and 9. Once again, it will be appreciated that the depiction of two different forms of bone anchor attached to the same vertebra is for illustration purposes only, and in practice it will generally be the case that pairs of bone anchors applied to any particular vertebra will be of the same form.

The fourth embodiment 41 has a baseplate 42 that is generally similar to that of the embodiments described above, having a generally concave undersurface that is shaped to fit against the pedicle of a vertebra with a congruent fit.

The fourth embodiment 41 differs from those previously described in the nature of the attachment part provided on the upper surface of the baseplate 42. In this embodiment 41, an eyelet 43 is formed integrally with the baseplate 42. As can be seen from FIG. 7, when the bone anchor 41 has been affixed to the vertebra, the eyelet 43 extends substantially perpendicularly from the patient's spine and is aligned essentially parallel to the spine. Bone anchors of this type applied to two or more vertebrae may be connected to rods by wires or the like tied to the eyelets 43.

The fifth embodiment 51, shown in FIGS. 7 and 9, is similar to that of FIG. 3, in that it has a baseplate 52 that is formed with a bushing 53 around an opening 54. The bushing 53 serves as a guide for insertion of a pedicle screw (not shown). Thus, the bone anchor 51 may be attached to the pedicle using adhesive, and then fastened more securely in place by means of the screw. Alternatively, the bone anchor 51 may be attached only with the screw.

As described above, the bone anchors such as those just described in detail may be produced by various methods, including both additive and subtractive manufacturing methods.

FIG. 10 illustrates schematically the manufacture of the bone anchor 31 of FIG. 5 by an additive process. Such a process involves the stepwise formation of the bone anchor 31 in a series of layers (“segments”) that are fused together. FIG. 10 shows the bone anchor 31 in a semi-complete state, with the next layers to be formed (“a” and “b”) shown separately. The layer “b” incorporates the first part of the ball-headed upstands 33,34, and those upstands and the remainder of the bone anchor 31 will be completed by the formation of further layers of fused material. Thus, the bone anchor 31 is progressively built up, layer by layer.

FIG. 11 is a schematic illustration of the manufacture of the bone anchor 31 by an alternative, subtractive, manufacturing method. In such a method, the bone anchor 31 is produced by machining, eg multi-axis milling, from a block of solid material 110. Thus, the bone anchor 31 emerges from the block 110, as material is machined away from the block 110.

Whichever manufacturing method is used, the general process is the same and includes the collection of medical data (ie the data file defining the form of the bone to which the bone anchor is to be applied), segmentation to define the series of layers necessary for creation of the undersurface of the baseplate (ie the bone abutment surface), determination of the optimal positions and orientation of any pedicle screw guides, selection and positioning of attachment features and areas of porosity, and the creation of a final CAD file. That CAD file is then used to control the manufacturing process. As described above, a number of bone anchors may be produced, each having the same bone abutment surface but having alternative attachment parts, so that the surgeon is provided with a range of alternatives from which he can choose prior to, or during, the surgical procedure.

In the first step, the bones are scanned to provide the necessary two-dimensional medical data that will be used to create a three-dimensional image. This will normally be CT or MRI with CT and will be provided by the radiologist working with the clinician. The next step is segmentation. Once a segmentation mask has been created, it is straightforward to convert it into a three-dimensional model. As this may be created using industry standard methods it does not require decisions by the technician. The design of the undersurface of the baseplate of the bone anchor is defined by the surface of the vertebra to which it will be attached. If a screw or attachment part needs to be positioned in line with the pedicle then computer techniques to visualise the screw and rotate the three-dimensional design may be used at this stage to ensure correct positioning and check whether the screw intersects with the outer bone of the vertebra. The attachments can then be chosen from a library of attachment parts, as can the position of any porosity and the size and arrangement of porous regions in the bone anchors. Once all the aspects of the bone anchor have been chosen, positioned and designed, the virtual model can be created in vectors and output in a standard file format, eg a CAD file, before final validation and sign-off by the clinician. The data file is then sent to the manufacturer who generally produces the bone anchors by additive or subtractive manufacturing.

In order to ensure optimal spacing any of the bone anchors of FIGS. 1 to 9 from the surface of the bone to which the bone anchor is applied, the bone abutment surface of the bone anchor may be formed with one or more downwardly depending spacer formations that bear against the surface of the bone and position the bone abutment surface at precisely the desired separation from the bone, the void between bone abutment surface and the bone thus being occupied by precisely the desired thickness of adhesive.

This is illustrated schematically in FIGS. 12(a) and 12(b), both of which are fragmentary cross-sectional views of a bone anchor according to the invention positioned on the surface of a bone.

Referring first to FIG. 12(a), there is shown the peripheral region of the baseplate 2 of the bone anchor 1 of FIGS. 1 and 2 (though the bone anchor could be any of the other illustrated embodiments, or indeed any other bone anchor according to the invention). The bone anchor 1 is positioned on the surface of a bone designated B.

As can be seen, the underside of the baseplate 2 (ie the bone abutment surface) has a form that mirrors the contours of the bone B. At its periphery, however, the baseplate 2 is formed with a downwardly depending rim 8 that bears directly on the surface of the bone B. The effect of the rim 8 is to position the bone abutment surface of the bone anchor 1 at a precise separation from the bone. That separation corresponds to the depth of the rim 8 (typically 80 μm or 100 μm). Because of the correspondence between the contours of the bone and the bone abutment surface, the void between them is of substantially uniform depth. That void is occupied by adhesive (designated A), the layer of adhesive thus also being of uniform thickness.

FIG. 12(b) is similar, but shows a pillar 9 that depends downwardly from the bone abutment surface. The effect of that pillar 9 is similar to that of the rim 8, positioning the bone abutment surface precisely relative to the bone surface and hence providing for a uniform and optimised thickness of adhesive A. The pillar 9 is depicted with a generally cylindrical form, but may have other forms, such as cuboid or conical.

Of course, a bone anchor according to the invention may be provided with more than one such spacer formation and indeed more than one type of spacer formation.

1. A bone anchor having a bone abutment surface adapted for congruent attachment to a bone.

2. The bone anchor of claim 1, wherein the bone anchor is adhered to the bone by means of adhesive interposed between the bone and the bone abutment surface.

3. The bone anchor of claim 2, wherein the adhesive is selected from the group consisting of acrylate or methacrylate adhesives, dentine bonding agents including but not limited to glass-ionomer composites, and bone cements.

4. The bone anchor of claim 1, which has a congruent fit with a part of the spine.

5. The bone anchor of claim 4, wherein the part of the spine is a vertebra.

6. The bone anchor of claim 5, wherein the part of the spine is the pedicle of a vertebra.

7. The bone anchor of claim 6, wherein the bone anchor is attached to the bone by means of a pedicle screw.

8. The bone anchor of claim 1, wherein the bone anchor is provided with one or more attachment parts for coupling the bone anchor to other components.

9. The bone anchor of claim 8, wherein the attachment parts are selected from the group consisting of an eyelet connection, a ball connection, a hole and bushing, and a drilling and/or tapping guide.

10. The bone anchor of claim 1, wherein the bone anchor includes a porous region.

11. The bone anchor of claim 1, wherein all or part of the bone anchor carries a coating including hydroxyapatite and/or cytokines.

12. A method for the production of a bone anchor, which method comprises:
manufacturing the bone anchor with a bone abutment surface formed in accordance with a data file embodying the form of the bone to which the bone anchor is to be attached, such that the abutment surface is adapted for congruent attachment to the bone.

13. The method of claim 12, which method comprises the preliminary step of generating the data file embodying the form of the bone to which the bone anchor is to be attached.

14. The method of claim 13, wherein the data file is the output of an imaging procedure.

15. The method of claim 14, wherein the imaging procedure is computer-tomography (CT), magnetic resonance imaging (MRI) or MRI with CT.

16. The method of claim 12, wherein the bone anchor is manufactured using an additive manufacturing process or a subtractive manufacturing process.

17. (canceled)

18. The method of claim 12, wherein the bone anchor is made from titanium or titanium alloy or polyetheretherketone (PEEK).

19. (canceled)

20. A plurality of bone anchors having bone abutment surfaces adapted for congruent attachment to the same area of a patient's bone, the bone anchors being provided with differing attachment parts for coupling of the bone anchor to other components.

21. The plurality of bone anchors of claim 20, wherein the attachment parts are selected from the group consisting of an eyelet connection, a ball connection, a hole and bushing, and a drilling and/or tapping guide.

22. A kit for use in spinal surgery, the kit comprising at least two bone anchors, each having a bone abutment surface adapted to have a congruent fit with a corresponding vertebra of a patient's spine, the bone anchors extending when applied to those vertebrae across both pedicles of the vertebrae, and the bone anchors being provided with attachment parts disposed centrally and adapted for coupling to a rod disposed, in use, substantially centrally of the patient's spine.

23. A surgical method, which method includes the step of affixing to a bone a bone anchor having a bone abutment surface adapted for congruent attachment to the bone,
wherein the bone anchor has been manufactured by a process including the steps of
a) using an imaging technique to generate a data file embodying the form of the bone to which the bone anchor is to be attached; and
b) manufacturing the bone anchor with a bone abutment surface formed in accordance with the data file, such that the abutment surface is adapted for congruent attachment to the bone.

24. The method of claim 23, which is a method for correcting spinal deformities, and includes the steps of affixing bone anchors to at least two vertebrae and then connecting the bone anchors to support the spine.

25. The method of claim 23, which is a method for fusing adjacent vertebrae in the spine and, includes the steps of affixing bone anchors to at least two adjacent vertebrae and then connecting the bone anchors to fuse the vertebrae.

26. The method of claim 23, wherein the bone anchor is adhered to the bone or vertebra by means of adhesive interposed between the bone and the bone abutment surface.

27. The bone anchor of claim 26, wherein the adhesive is selected from the group consisting of acrylate or methacrylate adhesives, dentine bonding agents including but not limited to glass-ionomer composites, and bone cements.

28. The method of claim 23, wherein the bone anchor(s) are provided with one or more attachment parts for coupling the bone anchor to other components.

29. The method of claim 28, wherein the attachment parts are selected from the group consisting of an eyelet connection, a ball connection, a hole and bushing, and a drilling and/or tapping guide.

30. The method of claim 23, wherein the bone anchor(s) include a porous region.

31. The method of claim 23, wherein all or part of the bone anchor(s) carries a coating including hydroxyapatite and/or cytokines.

32. (canceled)
